(19) United States
(12) Patent Application Publication
(10) Pub. No.: US 2003/0166511 A1
(43) Pub. Date: Sep. 4, 2003

(54) Cardioprotective composition comprising ceruloplasmin and uses thereof

(75) Inventors: Mircea Alexandru Mateescu, Montreal (CA); Joanne Paquin, Montreal (CA); Denis Langlois, Ste-Anne des Lacs (CA); Mhamed Aouffen, Montreal (CA)

Correspondence Address: DARBY & DARBY P.C., 805 Third Avenue, New York, NY, 10022, US

(73) Assignee: GESTILAB INC., Ste-Anne des Lacs (CA)

(21) Appl. No.: 10/021,691

(22) Filed: Nov. 5, 2001

Related U.S. Application Data
(63) Continuation of application No. PCT/CA00/00528, filed on May 5, 2000.

(30) Foreign Application Priority Data
May 5, 1999 (CA) 2,271,192

(51) Int. Cl. A61K 38/41; A61K 31/19; A61K 31/202

(52) U.S. Cl. 514/6; 514/557; 514/560

(57) ABSTRACT

A cardioprotective composition for protecting heart against oxidative stress and methods for using and preparing the same. More particularly, the cardioprotective composition of the invention comprises a mixture of pyruvate, antioxidant, fatty acids and ceruloplasmin. The cardioprotective composition could be used for the treatment of heart attack/failure, the treatment of ischemic cardiopathy, the conservation of heart before and during transplantation, and the treatment heart oxidative stress related conditions.

### BACKGROUND OF THE INVENTION

1) Field of the Invention

The present invention relates to the use of an amphiphilic antioxidative composition as cardioprotective agent and to methods for using and preparing the same. More particularly, the present invention pertains to the use of a formulation of pyruvate, antioxidant, lipid(s) such as fatty acids and ceruloplasmin (and/or derivatives thereof for protecting heart against oxidative stress.

2) Description of the Prior Art

Reactive oxygen species (ROS) are implicated in the development of many heart dysfunctions. For instance, ischemia/reperfusion insults to this organ are among the leading causes of mortality in America. These insults are caused by complete or partial local occlusions of vasculature and by trauma to heart, and also occur during handling of graft destined to heart surgery. Furthermore, evidence has been accumulated that oxygen free radicals (OFR) are, at least in part, responsible for specific damages and arrhythmias at reperfusion of ischemic heart. Therefore, lipid peroxidation of myocardial membranes by OFR, has been considered a potential mechanism of reperfusion arrhythmias. Interestingly, many studies have shown that inhibition of free radical accumulation during myocardial ischemia and reperfusion with OFR scavengers, antioxidant enzymes and spin-trap agents reduce the severity of reperfusion-induced arrhythmias.

Until now, no ideal therapeutic agent were known to protect heart against oxidant species associated with various types of oxidative stress and, at the same time, to present good antifibrillatory action and with less side effects in arrhythmias associated with the reperfusion of ischemic heart.

TRIAD is a combination of pyruvate, antioxidant and fatty acids. This composition has been patented in 1997 in the U.S. as a therapeutic wound healing compositions (U.S. Pat. No. 5,652,274). Several related U.S. patents have also been issued for covering the uses of TRIAD in antikeratolytic compositions (U.S. Pat. No. 5,641,814); in anti-fungal compositions (U.S. Pat. No. 5,663,208); in acne healing compositions (U.S. Pat. No. 5,646,190); in anti-inflammatory compositions (U.S. Pat. No. 5,648,380); in dermatological compositions (U.S. Pat. No. 5,602,183); in sunscreen compositions (U.S. Pat. No. 5,674,912); in antihistamine compositions (U.S. Pat. No. 5,614,561); in cytoprotective compositions (U.S. Pat. No. 5,633,285); in wound healing composition affixed to razor cartridges (U.S. Pat. No. 5,682,302); and in regenerating compositions (EP 0 573 465 B1). However, none of these patents disclose or suggest the use of TRIAD as cardioprotective and antifibrillatory agent.

Ceruloplasmin (CP), is a multifunctional blue-copper plasma protein which has important antioxidant properties as well as an oxidase and a ferroxidase activity. Ceruloplasmin was shown as an important oxygen free radical (OFR) scavenger. Recent studies related to the alterations in the level of ceruloplasmin further support the dominant role of this protein, suggesting possible therapeutic applications. For example, international patent application No WO9825954 relates to the use of modified ceruloplasmin comprising a glycosylphosphatidylinositol moiety and its use for the treatment of toxic level of ferrous iron. Although the cardioprotective effect of CP in conditions of oxidative stress has been shown (see Example at section 1.3), the synergistic cardioprotective action of CP when used in combination with an amphiphilic antioxidative composition comprising sodium pyruvate, antioxidant and fatty acids such as TRIAD, has never been disclosed and was therefore unexpected.

In view of the above, it is clear that there is a need for a partly lipidic/partly hydrophilic antioxidative composition comprising pyruvate, antioxidant(s), lipid(s) such as fatty acids and ceruloplasmin, to protect the heart against oxidant species and, at the same time, to provide antifibrillatory effects in arrhythmias associated with the reperfusion of ischemic heart. There is also the need for a cardioprotective composition wherein the compounds therein reciprocally enhance their respective cardioprotective effects.

The purpose of this invention is to fulfil these needs along with other needs that will be apparent to those skilled in the art upon reading the following specification.

### SUMMARY OF THE INVENTION

The present invention relates to a cardioprotective composition and more particularly to an amphiphilic antioxidative composition and its uses.

According to an aspect of the invention, the cardioprotective composition comprises a therapeutically effective amount of a mixture of pyruvate, antioxidant(s), lipid(s) and ceruloplasmin or a functional derivative thereof. These components are present in an amount that have a synergistic protective effect on cardiac cells.

In a preferred embodiment, lipids consist of a mixture of saturated and unsaturated fatty acids selected from the group consisting of monogylcerides, digylcerides, trigylcerides, free fatty acids, and mixtures thereof.

Advantageously, ceruloplasmin or its functional derivative is purified from blood using an one-step affinity chromatography on aminoethyl-agarose.

Preferably, pyruvate is selected from the group consisting of pyruvic acid, pharmaceutically acceptable salts of pyruvic acid, prodrugs of pyruvic acid, and mixtures thereof.

Preferably, also the antioxidant is selected from lipid-soluble antioxidants, and more preferably the antioxidant is selected from the group consisting of Vitamin A, carotene, Vitamin E, pharmaceutically acceptable salts thereof, and mixtures thereof.

According to an other aspect of the invention, the cardioprotective composition is used as such or as an active agent in the preparation of a medication for the treatment of heart and cardiac cells. Such treatments include the treatment of heart attack/failure, the treatment of ischemic cardiopathy, the conservation of heart before and during transplantation, and the treatment heart oxidative stress related conditions.

According to an other aspect of the invention, the invention provides a method for treating a heart oxidative stress related condition, the method comprising administrating to a patient in need thereof a therapeutically effective amount of an antioxidative composition comprising pyruvate, at least one antioxidant, at least one lipid and ceruloplasmin or a functional derivative thereof.

Alternatively, the invention also provides a method for treating a heart oxidative stress related condition comprising: a) administrating to a patient in need thereof, a therapeutically effective amount of an antioxidative composition comprising pyruvate, at least one antioxidant and ceruloplasmin or a functional derivative thereof; and b) providing, into the blood circulation of this patient, at least one lipid having a synergistic therapeutic effect on heart and cardiac cells with said antioxidative composition. The lipid(s) could be provided to the patient by increasing its lipidic blood level ratio through its diet. Examples of heart oxidative stress related conditions includes an heart attack/failure, ischemic cardiopathy, or handling an heart before and during an heart transplantation. According to an other aspect of the invention it is provided a method for preparing a cardioprotective composition, the method comprising the steps of:

a) providing a therapeutically effective amount of: i) pyruvate, ii) at least one antioxidant; iii) at least one lipid, and iv) ceruloplasmin or a functional derivative thereof; and

b) mixing together the components i), ii) iii) and iv) of step a) in a physiological buffered saline solution to obtain a homologous pharmaceutically acceptable suspension; and optionally

c) centrifuging or filtering the homologous suspension obtained in step b).

The buffered saline solution may comprises sodium, potassium, magnesium and calcium ions at physiological concentrations and if necessary, an emulsifier.

An advantage of the present invention is that it provides effective means for preventing the loss of viability and/or stimulates repair of heart and cardiac cells in conditions of oxidative stress. It can also protect heart from a toxic substance or a stress, stabilizes the cellular membrane of a heart or cardiac cell and/or helps in the normalization of cardiac cellular functions.

Other objects and advantages of the present invention will be apparent upon reading the following non-restrictive description of several preferred embodiments made with reference to the accompanying drawings.

### DESCRIPTION OF THE DRAWINGS

FIG. 1 is a diagram showing the time course protocol used for testing the composition of the invention.

FIG. 2 depicts in graphs the effect of various concentrations of ceruloplasmin on cardiodynamic variables (HB, CF and LVP) of isolated rat heart in presence of TRIAD S2 (0.5 X).

FIG. 3A is a bar graph showing the incidence of irreversible ventricular fibrillation (IVF) on the isolated heart under treatment with TRIAD (0.16 X), ceruloplasmin (CP; 0.5 μM) and their association.

FIG. 3B is a bar graph showing the relation treatment with TRIAD (0.16 X), ceruloplasmin (CP; 0.5 μM) and their association with respect to the cardioprotection.

### DETAILED DESCRIPTION OF THE INVENTION

As stated hereinbefore the present invention relates to the use of an amphiphilic antioxidative composition as cardioprotective agent. As disclosed herein, a composition comprising pyruvate, antioxidant, lipid(s) such as fatty acids and ceruloplasmin has synergistic cardioprotective actions against oxidative stress.

Unless defined otherwise, all technical and scientific terms used herein have the same meaning as commonly understood by one ordinary skilled in the art to which this invention belongs.

As used herein, the term “cardioprotective agent” or “cardioprotective composition” refers to any compound (or to any mixture of compounds) that protects heart from a toxic substance or a stress, stabilizes the cellular membrane of a cardiac cell and/or helps in the normalization of cardiac cellular functions. As used herein, the terms “cardiac cells” includes cells from the organ (mainly myocytes) as well as endothelial vascular cells. A “cardioprotective agent” thereby prevents the loss of viability and/or stimulates repair of cardiac cells and tissues. It will also preferably improve, at the organ level, the cardiodynamic variables (coronary flow, heart rate, left ventricular pressure) of the heart in conditions of oxidative stress.

Therefore, the term “cardioprotection” as used herein refers to the capacity of a cardioprotective agent to maintain the cardiodynamic variables at their normal level or to induce a fast recovery to the normal level, even in pathological or harmful conditions such as oxidative stress conditions including those occurring at post-ischemia reperfusion, inflammation.

As stated out above, the cardioprotective compositions of the invention comprises a mixture of (a) pyruvate; (b) at least one antioxidant; (c) at least one lipid such as fatty acids, preferably a mixture of saturated and unsaturated fatty acids; and (d) ceruloplasmin or a functional derivative thereof. According to the invention, these four components have a synergistic beneficial effect on cardiac cells, i.e. their combined effect is greater than the sum of their individual effects.

The pyruvate in the present invention may be selected from the group consisting of pyruvic acid, pharmaceutically acceptable salts of pyruvic acid, prodrugs of pyruvic acid, and mixtures thereof. In general, the pharmaceutically acceptable salts of pyruvic acid may be alkali salts and alkaline earth salts. Preferably, the pyruvate is selected from the group consisting of pyruvic acid, lithium pyruvate, sodium pyruvate, potassium pyruvate, magnesium pyruvate, calcium pyruvate, zinc pyruvate, manganese pyruvate, methyl pyruvate, α-ketoglutaric acid, and mixtures thereof. More preferably, the pyruvate is selected from the group of salts consisting of sodium pyruvate, potassium pyruvate, magnesium pyruvate, calcium pyruvate, zinc pyruvate, manganese pyruvate, and the like, and mixtures thereof. Most preferably, the pyruvate is sodium pyruvate.

The amount of pyruvate present in the cardioprotective composition of the present invention is a therapeutically effective amount. A therapeutically effective amount of pyruvate is that amount of pyruvate necessary for the cardioprotective composition to prevent and/or reduce injury of heart. The exact amount of pyruvate will vary according to factors such as the type of condition being treated as well as the other ingredients in the composition. Typically, the amount of pyruvate should vary from about 0.01 mM to about 100 mM. In a preferred embodiment, pyruvate is present in the composition of the cardioprotective perfusing solution in an amount from about 0.1 mM to about 20 mM, preferably from about 0.5 mM to about 10 mM. In the most preferred embodiment, the cardioprotective composition comprises about 2.5 mM of sodium pyruvate.

Antioxidants are substances which inhibit oxidation or suppress reactions promoted by oxygen, oxygen free radicals (OFR), oxygen reactive species (ORS) including peroxides. Antioxidants, especially lipid-soluble antioxidants, can be absorbed into the cellular membrane to neutralize oxygen radicals and thereby protect the membrane. The antioxidants useful in the present invention are preferably vitamin antioxidants that may be selected from the group consisting of all forms of Vitamin A including retinal and 3,4-didehydroretinal, all forms of carotene such as Alpha-carotene, β-carotene, gamma-carotene, delta-carotene, all forms of Vitamin C (D-ascorbic acid, L-ascorbic acid), all forms of tocopherol such as Vitamin E (Alpha-tocopherol, 3,4-dihydro-2,5,7,8-tetramethyl-2-(4,8,12-trimethyltri-decyl)-2H-1-benzopyran-6-ol), β-tocopherol, gamma-tocopherol, delta-tocopherol, tocoquinone, tocotrienol, and Vitamin E esters which readily undergo hydrolysis to Vitamin E such as Vitamin E acetate and Vitamin E succinate, and pharmaceutically acceptable Vitamin E salts such as Vitamin E phosphate, prodrugs of Vitamin A, carotene, Vitamin C, and Vitamin E, pharmaceutically acceptable salts of Vitamin A, carotene, Vitamin C, and Vitamin E, and the like, and mixtures thereof. Preferably, the antioxidant is selected from the group of lipid-soluble antioxidants consisting of Vitamin A, β-carotene, Vitamin E, Vitamin E acetate, and mixtures thereof. More preferably, the antioxidant is Vitamin E or Vitamin E acetate. Most preferably, the antioxidant is Vitamin E. Analogues of Vitamin E such as Trolox®, a compound which is more hydrosoluble than natural forms of Vitamin E and which could reach intracellular sites more rapidly, could also be used according to the present invention.

The amount of antioxidant present in the cardioprotective composition of the present invention is a therapeutically effective amount. A therapeutically effective amount of antioxidant is that amount necessary for the cardioprotective composition to prevent and/or reduce injury of the heart. The exact amount of antioxidant will vary according to factors such as the type of condition being treated as well as the other ingredients in the composition. Typically, the amount of antioxidant should vary from about 0.01 unit/ml to about 10 unit/ml. In a preferred embodiment, vitamin E antioxidant is present in the composition of the cardioprotective perfusing solution in an amount from about 0.01 unit/ml to about 2 unit/ml, preferably from about 0.05 unit/ml to about 1 unit/ml. In the most preferred embodiment, the cardioprotective composition comprises about 0.25 unit of antioxidant (α-tocopherol type VI in oil) per ml of cardioprotective composition.

As it is well known, lipids are esters or carboxylic acid compounds found in animal and vegetable fats and oils. The composition may comprises a single type of lipid or various types of different lipids. Preferably lipids are in the form of a mixture of saturated and unsaturated fatty acids. However, other types of lipids could be used such as glycolipids and phospholipids (e.g. lecithin). Lipid(s) or mixture thereof are selected among those lipids required for the stabilization or repair of the cellular membrane of cardiac mammalian cells. These lipids may be derived from animal or vegetables. In a preferred embodiment, selected lipids are in the form of mono-, di-, or triglycerides, or free fatty acids, or mixtures thereof, which are readily available for the stabilization or repair of the cellular membrane of cardiac mammalian cells. Artificial lipids which are soluble in organic solvents and are of a structural type which includes fatty acids and their esters, cholesterols, cholesteryls esters could also be used according to the present invention.

In a more preferred embodiment, the saturated and unsaturated fatty acids are those deriving from egg yolk. According to the use of the cardioprotective composition of the invention, replacing egg yolk as a source of fatty acids by chemical preparations of unsaturated, polyunsaturated and/or saturated fatty acids compatible with, and in proportions similar to those found in cell membranes, may be advantageous or reveal necessary to insure a controllable quality of preparations.

The amount of lipid(s) such as fatty acids present in the cardioprotective composition of the present invention is a therapeutically effective amount. A therapeutically effective amount of fatty acids for instance is that amount of fatty acids necessary for the cardioprotective composition to prevent and/or reduce injury of a cardiac tissue, without being toxic to cardiac cells. The exact amount of lipid(s) or fatty acids will vary according to factors such as the type of condition being treated as well as the other ingredients in the composition. Typically, the amount of lipid(s) or fatty acids should vary from about 0.001% v/v to about 1% v/v. In a preferred embodiment, fatty acids are present in the composition of the cardioprotective perfusing solution in an amount from about 0.001% v/v to about 0.2 v/v, preferably from about 0.005% v/v to about 0.1% v/v, by weight of cardioprotective composition. In the most preferred embodiment, the cardioprotective composition comprises about 0.025% v/v of fresh egg yolk.

As the lipidic blood level of an individual is normally about 0.5-0.6% of the total serum volume, the lipidic portion could be omitted from the cardioprotective composition of the invention. It could be possible to provide into the blood circulation of this individual at least one lipid having a synergistic therapeutic effect on cardiac cells with the others component of the antioxidative cardioprotective composition of the invention. For instance, selected lipid(s) could be provided by increasing the lipidic blood level ratio of this individual through the diet. Lipids which could have a synergistic therapeutic effect without being harmful to a patient could be selected from the group consisting of phospholipids, glycolipids, fatty acids, and mixture thereof.

As stated previously, ceruloplasmin (CP), is a multifunctional blue-copper plasma protein whose most known function is the copper transport. Ceruloplasmin also has important antioxidant and free radical scavenging properties as well as a ferroxidase I activity. Ceruloplasmin was also shown as an important oxygen free radical (OFR) scavenger. Another important role has recently been postulated for this protein as a regulator of iron metabolism.

The ceruloplasmin useful according to the present invention comprises substantially pure ceruloplasmin generally purified from blood or produced by recombinant techniques and functional derivatives thereof. As generally understood and used herein, the term substantially pure refers to a ceruloplasmin preparation that is generally lacking in other blood components.

A “functional derivative”, as is generally understood and used herein, refers to a protein sequence that possess a functional biological activity that is substantially similar to the biological activity of a particular protein sequence. A functional derivative of a protein may or may not contain post-translational modifications such as covalently linked carbohydrate, if such modification is not necessary for the performance of a specific function. The term “functional derivative” is intended to the “fragments”, “segments”, “variants”, “analogs” or “chemical derivatives” of a particular protein.

The terms “fragment” and “segment” as is generally understood and used herein, refers to a section of a protein, and is meant to refer to any portion of the amino acid sequence.

The term “variant” as is generally understood and used herein, refers to a protein that is substantially similar in structure and biological activity to either the protein or fragment thereof. Thus two proteins are considered variants if they possess a common activity and may substitute each other, even if the amino acid sequence, the secondary, tertiary, or quaternary structure of one of the proteins is not identical to that found in the other.

The term “analog” as is generally understood and used herein, refers to a protein that is substantially similar in function to ceruloplasmin.

As used herein, a protein is said to be a “chemical derivative” of another protein when it contains additional chemical moieties not normally part of the protein, said moieties being added by using techniques well know in the art. Such moieties may improve the protein's solubility, absorption, biodisponibility, stability, biological half life, and the like. Any undesirable toxicity and side-effect of the protein may be attenuated and even eliminated by using such moieties. For example, CP and CP fragments can be covalently coupled to biocompatible polymers (polyvinyl-alcohol, polyethylene-glycol, etc) in order to improve stability or to decrease antigenicity. They could also be coupled to proteins known to pass the blood-brain barrier via transcytosis across vascular endothelial cells (eg. transferrin).

The amount of ceruloplasmin and/or functional derivatives thereof present in the cardioprotective composition of the present invention is a therapeutically effective amount. A therapeutically effective amount of ceruloplasmin is that amount of ceruloplasmin or derivative thereof necessary to synergistically (in combination with the other components of the composition) prevent and/or reduce injury of heart. The exact amount of ceruloplasmin and/or functional derivatives thereof to be used will vary according factors such as the protein's biological activity, the type of condition being treated as well as the other ingredients in the composition. In a preferred embodiment, ceruloplasmin is present in the composition of the cardioprotective perfusing solution in an amount from about 0.05 μM to about 10 μM, preferably from about 0.1 μM to about 2 μM. In the preferred embodiment, the cardioprotective composition comprises about 0.5 μM of active ceruloplasmin.

Further agents can be joint to the cardioprotective composition of the invention. For examples various antioxidants may complete the action of the cardioprotective composition such as:

metal chelators/scavengers (e.g. desferrioxamine [Desferal®], a small substance capable to scavenge Fe³⁺ and other metal ions);

proteins or their fragments that can bind metal ions such as ferritin or transferrin which both bind Fe³⁺;

small scavengers of .O₂⁻ (superoxide), .OH (hydroxyl) or NO (nitric oxide) radicals (e.g. acetyl salicylic acid, scavenger of .O₂⁻; mannitol or captopril, scavengers of .OH; arginine derivatives, inhibitors of nitric oxide synthase which produce NO);

proteins or their fragments that scavenge OFR and can assist the protective action of ceruloplasmin (e.g. superoxide dismutase which dismutate .O₂⁻; hemoglobin which traps NO); and

proteins or their fragments that can scavenge H₂O₂ (hydrogen peroxide) in cases where they may exert a more potent or durable protective action than pyruvate (e.g. catalase, glutathion peroxidase).

The compositions of the invention may also comprises modulators of heart functions such as hormones, trophic factors, or analogs of these substances that act by binding to heart receptors (e.g. ligands of β-adrenergic receptors in cardiac arrhythmias.

Further to the therapeutic agents, the cardioprotective composition of the invention may also contain preserving agents, solubilizing agents, stabilizing agents, wetting agents, emulsifiers, sweeteners, colorants, odorants, salts, buffers, or coating agents. For preparing the cardioprotective composition, methods well known in the art may be used.

The method of preparation of the cardioprotective compositions of the invention consist simply in the mixing of components in a buffered saline solution in order to get a homogenous suspension. Suitable saline solution comprises sodium, potassium, magnesium and calcium ions at physiological concentrations, has an osmotic pressure varying from 280 to 340 mosmol, and a pH varying from 7.0 to 7.4. Depending of the amount and of type of lipid(s) which is used, the saline may also comprises an emulsifier. Preferably, the buffered saline solution is selected from the group consisting of modified Krebs-Henseleit buffer (KH) and phosphate buffer saline (PBS), both at pH 7.4. The homogenous suspension obtained can further be centrifuged and/or filtered to reduce its viscosity and/or eliminated non-soluble particles.

Obviously, this simple method can be modified according to the use of the cardioprotective composition. For example, in the example found hereunder, genuine and centrifuged-filtered preparations were used. However, it is important to note that modifications in the modality of preparation can influence the resulting effects of the cardioprotective compositions. For example, varying the pH of the composition (or buffer) can slightly modify the ionization state of carboxylic functions of pyruvate and thus alter its solubility and/or reaction with H₂O₂ while the dialysis of the composition would reduce the amount of pyruvate in the final preparation, unless it is done before addition of pyruvate. A person skilled in the art will know how to adapt the preparation of the cardioprotective composition of the invention according to their use in specific conditions in order to obtain positive effects.

The cardioprotective composition of the invention is suitable to treat diseases and pathological conditions such as heart attack/failure, heart diseases (ischemic cardiopathy), and in addition diseases involving copper metabolism (Wilson's and Menkes's diseases) and iron metabolism diseases (hemosiderosis, aceruloplasminmia). The protective composition of the invention could also be used during the handling of organs in transplantation (conservation of organs before and during transplantation, post-surgery survival). These cardioprotective compositions could also be involved in the treatment of diseases which were shown to involve oxidative stress conditions such as hepatitis, in the treatment of poisoning or the diminution of side effects of various drugs (such as chemotherapeutic and immunosuppressive drugs) especially in cases if deleterious action of various toxicants and drugs is exerted via production of reactive oxygen species.

The cardioprotective composition of the invention has potential applications in both fast (in minutes; especially for pyruvate) and long term treatments (hours and days; for antioxidant, lipid(s) and ceruloplasmin). The amount to be administered is a therapeutically effective amount. A therapeutically effective amount of a cardioprotective composition is that amount necessary for protecting heart from a toxic substance, stabilizing the cellular membrane of cardiac cells and/or helping in the normalization of cardiac cellular functions. Suitable dosages will vary, depending upon factors such as the type and the amount of each of the components in the composition, the desired effect (fast or long term), the disease or disorder to be treated, the route of administration and the age and weight of the individual to be treated.

The cardioprotective composition of the invention and/or more complex pharmaceutical compositions comprising the same may be given via various route of administration. Ways that can be considered are rectal and vaginal capsules or nasally by means of a spray. They may also be formulated as creams or ointments for topical administration. They may also be given parenterally, for example intravenously, intramuscularly or sub-cutaneously by injection or by infusion. Intravenous administration can be a way for fast answer in various clinical conditions (e.g. stroke and heart attacks, post-surgery treatments, etc). Obviously, the cardioprotective composition of the invention may be administered alone or as part of a more complex pharmaceutical composition according to the desired use and route of administration. Anyhow, for preparing such compositions, methods well known in the art may be used.

The cardioprotective composition could be administered per Os (e.g. capsules) depending of their composition i.e. to do so all composition's components must be absorbable by the gastrointestinal tract. For example CP as such cannot be recommended for oral administration because, as a large molecule, it would not be intestinally absorbed. This may not however apply to smaller and/or functional derivatives of this protein provided their formulation in absorbable forms (e.g. liposomes). Intravenous injection/perfusion and nasal sprays are possible ways to administer the compositions of the invention.

As it will now be demonstrated by way of an example hereinafter, the composition of the invention possesses a strong cardioprotective activity i.e. the capacity to maintain the cardiodynamic variables at their normal level or to induce a fast recovery to the normal level, even in pathological or harmful conditions such as oxidative stress conditions including those occurring at post-ischemia reperfusion fibrillation. Although any methods and materials similar or equivalent to those described herein can be used in the practice or testing of the present invention, the preferred methods and materials are described.

### EXAMPLE

### Synergistic Cardioprotective Actions of TRIAD and Ceruloplasminagainst Oxidative Stress

### Abstract

Oxidative stress, in particular that induced by ischemia and reperfusion, remains a major cause of acute heart injuries, leading to cardiac dysfunctions. It has been shown previously that Ceruloplasmin (CP), a multifunctional blue-copper plasma protein which has important antioxidant and free radical scavenging properties as well as a ferroxidase I activity, protects ischemic isolated rat heart against fibrillations due to reperfusion. In this study, the heart model was used to determine whether association of TRIAD and CP provides higher protection against oxidative stress damages than that observed for each agent alone. Heart-resistance to injury caused by ischemia-reperfusion was assessed by measuring occurrence of irreversible fibrillations. It was interesting to note that combination of suboptimal cardioprotective concentrations of CP and TRIAD offered more than additive protection to isolated hearts submitted to ischemia-reperfusion, suggesting synergistic effects. In conclusion, these results indicate that combination of CP and TRIAD provides higher antioxidant protection to heart than each agent alone, and suggest that enhanced protection can result from complementary spectrum of antioxidant properties.

### 1. Introduction

1.1. Ceruloplasmin—A Multifunctional Copper Protein

Ceruloplasmin (CP) is an important plasma blue-copper protein (α₂-globulin) with a multifunctional role (Gutteridge and Stocks, 1981). First of all, CP is the main copper carrier. As oxidase (EC 1.16.3.1) CP is involved in the regulation of biogenic amines and phenols level. Also known as Ferroxidase I, CP catalyses the Fe²⁺-->Fe³⁺ reaction (an important reaction considering the high toxicity of Fe²⁺). Ceruloplasmin was also shown as an important oxygen free radical (OFR) scavenger. Recently, CP was shown to be involved in angiogenesis, in relation with its function as copper carrier. Previously, the Inventors, in collaboration with Dr. Rui Wang of University of Saskatchewan, have discovered several unexpected physiological functions of copper proteins. The studies on these new functions, as cardioprotective and antifibrillatory actions, as well as the discovery of modulation of ionic channels in neurons, contributed to the knowledge on the CP biochemical and physiological roles, interesting for their possible therapeutic applications.

1.1.1. Ceruloplasmin and Human Pathology

Several diseases (Menkes, Wilson) are related to major alterations in the level of CP. Evolution of these diseases appears related to the CP level, in particular with the holo-CP (CP completely loaded with copper), underlining the essential role of CP as copper carrier. On the other hand, there are more and more data suggesting that oxidative stress is a factor in Parkinson, Alzheimer and other neurodegenerative diseases, whose evolution could be influenced by level or iron in brain. The possible involvement of this metal in neurodegenerative diseases, particularly in Parkinson disease, and the recent association of systemic hemosiderosis (aceruloplasminmia) with a mutation in the human CP gene, support the idea that the dominant role of this protein is that of a ferroxidase.

1.1.2. Ceruloplasmin Biochemistry

The “blue copper” center of CP has a characteristic absorption band at 610 nm and a two-copper pair is diamagnetic detectable and another copper is EPR (electronic paramagnetic resonance) detectable. Ceruloplasmin contains six copper atoms per molecule. Three copper atoms are aggregated in a cluster which is the Blue-Copper center of CP. Two others form a diamagnetic pair. The last one is paramagnetic (EPR detectable).

An absorbency ratio A_{610 nm}/A_{280 nm}=0.040 was considered in the literature as characteristic of a homogeneous standard pure enzyme with a proper conformation. It was reported for CP a high susceptibility at proteolysis, and physiological properties influenced by the molecular integrity. Despite intensive research in various laboratories, many aspects of CP are still unclear. The protein has been the object of many controversies (originated from its high susceptibility to proteolysis) concerning the molecular characteristics and the copper content. Also controversial was its complex physiological role (antioxidant/prooxidant). Within the last decade, a continuously growing interest concerns the molecular mechanisms of protection at cellular and tissular level, induced by CP.

It was recently shown that CP structure consists in six domains. Surprisingly, its configuration appears close to that of clotting Factor VIII. However, the enigma is not ended. The intriguing fact is that CP receptors were identified, localized in tissues strongly involved in oxidative processes (heart) or sensitive to oxidative stress (brain: known to be damaged by the oxidative stress, especially in aging). It is now established the presence of specific CP receptors, with specific localization on aorta and heart (Stevens et al, 1984), brain, erythrocytes and recently reported, on placenta. Liver endothelium was shown to bind, transport and desialate CP, which is then recognized by galactosyl receptors of hepatocytes. Also it was shown the secretion of CP by lung, brain (astrocytes), etc. What is the real role of this non circulating CP, is still to elucidate.

A questionable aspect is if CP (132 kDa) can be internalized as the whole molecule or as fragments. Chudej et al (1990) reported the transcytosis of exogeneous Superoxide Dismutase (SOD) and even of catalase (240 kDa) from coronary capillaries into dog myocytes. This is a particular case and a complete answer is not yet available. In any case, an interaction CP-cells was supposed. Possibly only copper is internalized.

1.2. ROS Scavenging Capacities of Ceruloplasmin in vitro

It was found that CP had better antioxidant and cardioprotective capacities than SOD (Dumoulin et al, 1996). Furthermore, CP was compared, in terms of antioxidant potential in vitro, with other well established antioxidants, using β-phycocyanin as a fluorescent indicator protein (Anastasiu et al., 1998). It was found, again, that CP exhibits a better scavenging capacity than SOD and than deferoxamine (Desferal™, an antifibrillatory agent acting as an iron chelating agent). The concentrations of CP ensuring good antioxidative activity in vitro was for the range 2-15 μM (Atanasiu et al., 1998), while Albumin as control exhibited a similar antioxidative action at a concentration much higher (260 μM).

1.3 Cardioprotective and Cardiomodulatory Actions of Ceruloplasmin Copper-Protein

Some of the present inventors (in a collaborative project with Dr. Réginald Nadeau at the Research Center of the Sacré-Coeur Hospital and Université de Montreal) were the first to show a cardioprotective effect of CP in conditions of oxidative stress (Chahine et al, 1991; Mateescu et al, 1995; Dumoulin et al, 1996), on the Langendorff model of rat isolated heart submitted to electrolysis induced ROS (Reactive Oxygen Species). Furthermore, it was shown an antifibrillatory effect of CP at reperfusion of ischemic isolated heart (Atanasiu et al, 1995). This aspect is important because the ischemia-reperfusion model is closely related to heart pathology. In part, the mechanism of cardioprotection can be explained by antioxidant properties of CP, limiting the damages at reperfusion (which is associated with an important oxidative stress). However, CP was shown to behave as a Class III antiarrhythmic drug, inducing a prolongation of Effective Refractory Period (ERP) and of the action potential (AP), in conditions without oxidative stress (Atanasiu et al, 1996). This means that some other properties of CP are involved in cardioprotection.

1.4 A Novel Single-Step Chromatographic Method for the Fast Ceruloplasmin Purification

Recently, a novel single-step chromatographic method have been reported for the fast CP purification, a method leading to a purified, electrophoretically homogeneous CP (Wang et al, 1994; Mateescu et al, 1999). Ceruloplasmin is susceptible to proteolytic denaturation and this fast method therefore protects CP against such denaturation by decreasing time of eventual contact with proteolytic enzymes found in plasma or blood. The purification procedure is based on the highly selective retention of CP on the Amino-ethyl (AE)-agarose (see Mateescu et al 1999, for details concerning the CP purification schema). Using this procedure, it is possible to obtain CP preparations with ratio A₆₁₀/A₂₈₀=0.045-0.070 and a very high oxidasic activity. Minimizing the sample story, the risk of protein degradation is limited. In fact it is supposed, following a reexamination of CP spectral properties (EPR [Calabrese et al, 1988]), that CP purified using this procedure is closer to its real native structure than commercial CP obtained by other methods. This method allows to realize an original CP immobilization. The conjugation of CP with biocompatible polymers is important because the immobilized enzyme conjugates show sought-for advantages such as higher stability, lower antigenicity and possibility to continuous use in various devices of potential interest for bioimplants or for organ preservation in view of transplantation.

1.5 Cardioprotective Action of TRIAD

As stated herein before, TRIAD is a combination of sodium pyruvate, antioxidant and fatty acids for which many uses have been patented. Preferably, TRIAD comprises sodium pyruvate, Vitamin E and egg yolk. Although this combination is also known under the name of CRT (Cellular Resuscitation Therapy), the current denomination of TRIAD is used herein. The three components were shown to act synergistically to ameliorate wound healing (Martin, 1996; Sheridan et al., 1997) and to reduce oxidative damage to keratinocytes and monocytes exposed to ultraviolet light (Martin, 1996) or to hepatocytes treated with doxorubicin (Gokhale et al., 1997). As shown hereinafter, TRIAD offers antioxidant protection to isolated hearts perfused with electrolyzed buffer or subjected to partial ischemia and reperfusion. In addition, despite a totally different composition of TRIAD versus CP, it was found to exert antifibrillatory properties on heart with results in certain extent, similar to those of CP.

1.6 Presentation of the Study

The objective of this study was to evaluate the cardioprotective action of CP in combination with TRIAD in order to determine if superior protection can be obtained by using antioxidants that could exert complementary actions. In this work, oxidative stress was achieved by subjecting isolated rat hearts to partial ischemia and reperfusion.

### 2. Materials and Methods

Materials

Vitamin E (α-tocopherol type VI in oil), sodium pyruvate, ethylenediamine tetraacetic acid (EDTA), and N,N-diethyl-p-phenylenediamine (DPD), and xanthine (XA) were purchased from (Sigma Chem. Co). Fresh egg yolk was the source of fatty acids. The other current chemicals were reagent grade (from Sigma Chem. Co., St-Louis) and were used without further purification.

Animals

Adult male Wistar rats (225-250 g) were from Charles River Inc. (Canada).

Methods

2.1 Preparation of Ceruloplasmin

Ceruloplasmin was purified from bovine plasma as already described (Wang et al, 1994; Mateescu et al, 1999), using a single affinity chromatography on aminoethyl-agarose. The value of A_{610 nm}/A_{280 nm} was approximately 0.045 for all preparations used in this study. Ceruloplasmin was stored at −20° C. in 0.1 M potassium phosphate buffer, pH 7.4, until use. Ceruloplasmin was used in its storage buffer to be injected in the perfusion buffer of isolated hearts.

2.2 Preparation of TRIAD and TRIAD (S2)

The 1×TRIAD concentration was prepared as Gokhale et al. (1997) and contained 0.1% v/v fresh egg yolk, 1 unit/ml vitamin E (α-tocopherol type VI in oil) and 10 mM sodium pyruvate. Stock 5× (5 fold) or 10× (10 fold) concentration of TRIAD was freshly prepared before each experiment by carefully mixing the three agents to get a homogenous suspension. TRIAD mixtures were made in a modified Krebs-Henseleit (KH) buffer (118 mM NaCl, 25 mM NaHCO₃, 3.8 mM KCl, 1.2 mM KH₂PO₄, 1.2 mM MgSO₄, 2.5 mM CaCl₂, 11 mM dextrose, pH 7.4). Pyruvate and vitamin E are soluble in egg yolk and miscible with both saline physiological buffers.

In an other study, the Applicant found that TRIAD was not compatible with the organ functions. Therefore, the genuine TRIAD preparations were modified as follows: 5× or 10×genuine preparations were centrifuged at 15 000×g for 20 min, at 4° C., and the resulting supernatants (S1) filtered on Whatman paper filter #54. The final filtered supernatant was named TRIAD (S2) and used to perfuse hearts. The different concentrations of TRIAD (S2) preparation were obtained by subsequent dilution with KH buffer (i.e. TRIAD (S2) 1× was obtained by 10 fold dilution of stock TRIAD (S2) 10×preparation). These supplementary steps yield in a less cloudy and less viscous preparations which were non toxic to the heart.

2.3 Studies on Isolated Rat Heart Submitted to Ischemia-Reperfusion

All experiments were conformed to rules of the Guide for the care and use of laboratory animals published by the US National Institutes of Health (NIH publication No 85-23, revised 1985). Adult male Wistar rats (225-250 g) were anaesthetized with sodium pentobarbitone intra-peritoneally (0.1 ml/100 g body weight) and then heparinized (500 UI intra-peritoneally). Hearts were rapidly excized, placed in ice-cold oxygenated modified Krebs-Henseleit (KH) buffer (a solution of 118 mM NaCl, 25 mM NaHCO₃, 3.8 mM KCl, 1.2 mM KH₂PO₄, 1.2 mM MgSO₄, 2.5 mM CaCl₂ and 11 mM dextrose, maintained to pH 7.4 by continuous gassing with a mixture of 95% O₂ et 5% CO₂), cleaned and then mounted on a modified Langendorff heart perfusion apparatus. Hearts were cannulated via the aorta and retrogradely perfused a constant perfusion pressure (90 mm Hg at 37° C.) with modified KH buffer. This solution was continuously gassed with a mixture of 95% O₂ and 5% CO₂ (to maintain a pH of 7.4), at 37° C. by constant temperature circulation (with water jackets around the pressurized arterial reservoir). In order to avoid precipitates, the perfusion buffer was filtered through a 5.0 μm cellulose acetate membrane to remove particulate contaminants. Hearts were perfused with KH buffer until equilibration (˜10 min)and then submitted to partial ischemia-reperfusion as described hereinafter.

Ischemia-Reperfusion

Hearts were perfused for a 10 min control period with KH buffer, and then 510 min with KH+TRIAD for stabilization. Regional ischemia was induced by occluding the left anterior descending artery with a tight ligature positioned around and at a point close to its origin, with a piece of plastic tubing. The resulting arterial occlusion that produces regional (partial) ischemia and consequently a reduction in coronary flow of 40%-50%, was maintained for 10 min. In fact, an acceptable regional ischemia was confirmed, in addition to the mentioned CF reduction, by 60-70% LVEDP elevation and by 40-50% LVP reduction. At the end of this 10 min arterial occlusion period, reperfusion was initiated by cutting the ligature with a scalpel bled and rhythm disturbances were monitored for 15 min more. Left ventricular pressure and epicardial ECG were continuously monitored before and during ischemia and reperfusion. Equilibrating perfusion, ischemia and reperfusion were all performed at 37° C.

Experimental Protocol

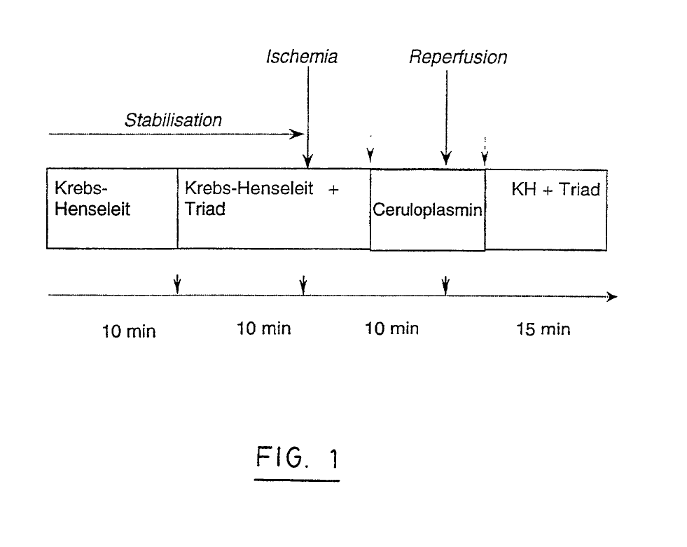

Hearts, under perfusion with TRIAD (S2) 0.5×, were treated with CP in concentrations from 0.5-4 μM. TRIAD was present in the perfusion buffer during equilibration, while CP was injected as a bolus in the perfusion buffer (not containing or containing TRIAD), just prior it entered heart. After treatment with each concentration of CP, cardiodynamic variables (HR, CF and LVP) were recorded for at least 2 min. Between the successive CP concentrations, the hearts were perfused with KH buffer containing TRIAD only, in order to wash-out the CP from each of the previous treatment. The experiment was repeated twice. The time course protocol depicted in FIG. 1 indicates duration of each step and the time of administration of TRIAD and CP. Ceruloplasmin was administered at a middle of ischemia period and stopped 2 min after perfusion.

Recorded Cardiodynamic Variables

The cardiodynamic variables: left ventricular pressure (LVP), heart rate (HR) coronary flow (CF) and epicardial electrogram (ECG), were monitored as follows. Briefly, a saline-filled latex balloon was inserted into the left ventricle by way of the AV valve and connected via a polyethylene cannula to a pressure transducer for determination of Left Ventricular Pressure (LVP) and Left Ventricular End Diastolic Pressure (LVEDP). The intraballoon volume was adjusted to exert a physiologic LVEDP of 10 mm Hg. Epicardial electrogram (ECG) was obtained using two silver electrodes, one inserted into the ventricular apex, and the other connected to the aortic cannula. The LVP, LVEDP, and ECG were recorded on a Nihon-Kohden polygraph (RM 600); heart rate (HR) was calculated from the electrogram. Coronary flow (CF) was measured by time collection of coronary effluent volume at various times during the experiment.

Rat hearts were first perfused for 10 min with KH buffer and then for another 10 min with the same buffer containing TRIAD (S2) prepared as described above (section 2.2), until equilibration of cardiodynamic variables was achieved. Perfusion with buffer containing TRIAD(S2) was pursued during 10 min of partial ischemia of the heart and continued during 10 min of the reperfusion. The cardioprotective effect of TRIAD and CP was investigated in reperfusion of regional ischemia isolated rat hearts after regional ischemia, under treatment with TRIAD (S2) 0.16×, with and without 0.5 μM CP associated to the treatment. FIG. 1 depicts the protocol used for treatment of ischemic heart with TRIAD and CP. Ceruloplasmin was administered at a middle of ischemia period and stopped 2 min after reperfusion. Details on the experimental conditions for the ischemia and reperfusion are presented hereinabove. Left ventricular pressure and epicardial ECG were continuously monitored before and during ischemia and reperfusion.

Heart in the control group (n=12) were perfused with KH buffer throughout the experiment and submitted to 10 min partial ischemia without any cardioprotective treatment.

Quantification of Arrhythmia

Arrhythmia were defined according to the Lambeth convention (Walker et al., 1988). ECG recordings were analyzed for the incidence of irreversible ventricular fibrillation (IVF) and for the time of normal sinus. It was analyzed whether fibrillation was spontaneously reversible, or hearts remained in irreversible ventricular fibrillation (more than 120 seconds). Ventricular fibrillation was defined as a ventricular rhythm with no recognizable QRS complex and with an amplitude less than of the normal electrogram. In addition, the total time during which each heart remained in normal sinus rhythm during the first 5 min of reperfusion, was quantified.

Statistical Analysis

With the exception of incidences of arrhythmias (calculated in percentage of fibrillating hearts, reported to the total number of hearts in experiment), all results were expressed as mean (±SEM).

### 3. Results

3.1 Cardioprotection Afforded by CP+TRIAD (S2) Against Ischemia-Reperfusion Injury on Isolated Rat Heart

Reperfusion of ischemic heart generates drastic damages. Control heart (in the absence of cardioprotection) exhibited 100% irreversible fibrillation. The total duration of normal sinus rhythm over the 5 minutes of reperfusion was extremely short, only 25 sec.

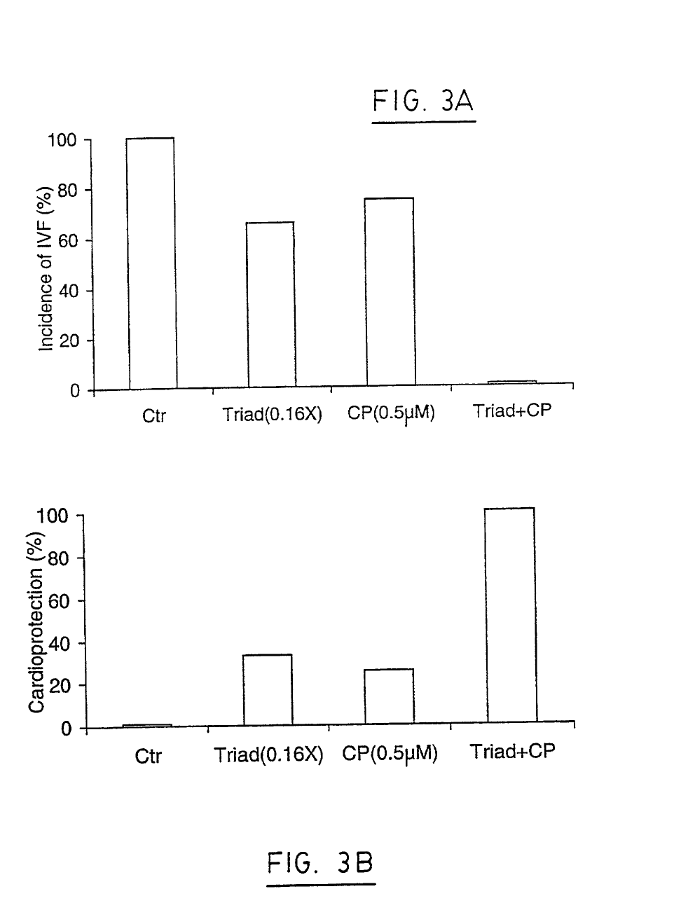

FIG. 3 shows that TRIAD (S2) 0.16× (suboptimal concentration) reduced the incidence of reperfusion-induced irreversible ventricular fibrillation (IVF) from 100% to 66% (cardioprotection of 34%), while CP (0.5 μM) generated a decrease 100% to 75% (cardioprotection of 25%). At the same time, unexpectedly, the association of TRIAD (0.16×) and CP (0.5 μM), totally reduced the incidence of IVF at reperfusion from 100% to 0% (cardioprotection of 100%; FIG. 3). This cardioprotection afforded by the association of both therapeutic agents, is definitely higher than the sum (59%) the cardioprotection values afforded by each one of the two agents (FIG. 3). These surprising data suggest that the association of TRIAD and CP presents a synergistic cardioprotection.

### 4. Discussion

As mentioned above, results showed that TRIAD and CP synergistically afford antifibrillatory protection of ischemic heart at reperfusion (FIG. 3). It is thus astonishing to find that a partial lipophilic/partial hydrophilic antioxidative composition comprising TRIAD (sodium pyruvate, vitamin E, egg yolk fatty acids) and CP cause a synergistic enhancement of the cardioprotective effects of each of these compounds.

A possible explanation of this synergistic action, can be based on the reciprocal modulation of membrane effects of CP and TRIAD. Heart receptors for CP have previously mentioned (Stevens et al, 1984). On the other hand, TRIAD, with fatty acids and vitamin E in its composition, also would act on membrane. It appears that from both actions, of CP and of TRIAD, a synergistic enhancement of cardioprotection can occur. In fact, during early reperfusion of ischemic myocardium, the influx of oxygen in presence of metabolic intermediates accumulated during the ischemic period, will generate OFR, exceeding the antioxidative capacity of the tissue. Oxygen free radicals, in particular the hydroxyl radical, may exacerbate ischemia induced injury by promoting oxidative modifications in cell membrane phospholipids, enzymes and ionic pumps.

For the cardioprotective effects of TRIAD it was supposed a mechanism related to its three components. Pyruvate, able to enter the cell, will enhance intracellular defense, while vitamin E and egg yolk will improve membrane functionality (Martin, 1994, 1996).

In case of antioxidative defense, it is possible that both TRIAD and CP (Chahine et al, 1991; Mateescu et al, 1995; Atanasiu et al, 1995), as antioxidants, will probably limit the leakage of cellular Fe²⁺ ion (easily generated by reduction of Fe³⁺→Fe²⁺, induced by superoxide anion which is a reductive agent), preventing thus the production of hydroxyl radical (.OH) via the Fenton and Haber-Weiss reactions. Mechanisms of iron involvement are not fully elucidated, but there is a growing consensus that oxidative tissue damage is related to non-heme cellular iron mobilized from cytosolic metal-containing sites: e.g. myoglobin and ferritin stores within endothelial and myocardial cells. Most of intracellular iron is deposited in ferritin (which can store 2000 up to 4500 of Fe³⁺ ions per complex) from where it can be released and, in the presence of reducing equivalents (e.g. superoxide radicals), is reduced in the ferrous (Fe²⁺) form. This may explain the toxicity of superoxide anion. The initial damage results in a generalized release of iron (Fe²⁺) into the cellular environment, and more widespread nonspecific injury may result. Although TRIAD, CP and other cardioprotective antioxidants (i.e. deferoxamine, an iron-chelating agent) act by different mechanisms, their ultimate protective effects are probably exerted by the same prevention of ROS. The antioxidant capacity would explain an additive effect of CP on TRIAD cardioprotection. The synergistic effect of CP on the TRIAD cardioprotection, can reside in the fact that, in addition to its antioxidant capacity, CP, if retained on cells binding proteins or receptors (Stevens et al, 1984), will exert, in situ: i) its ferroxidase action oxidizing ferrous ions released by outside diffusion and ii) scavenging superoxide radicals and reducing thus the formation of hydroxyl radicals.

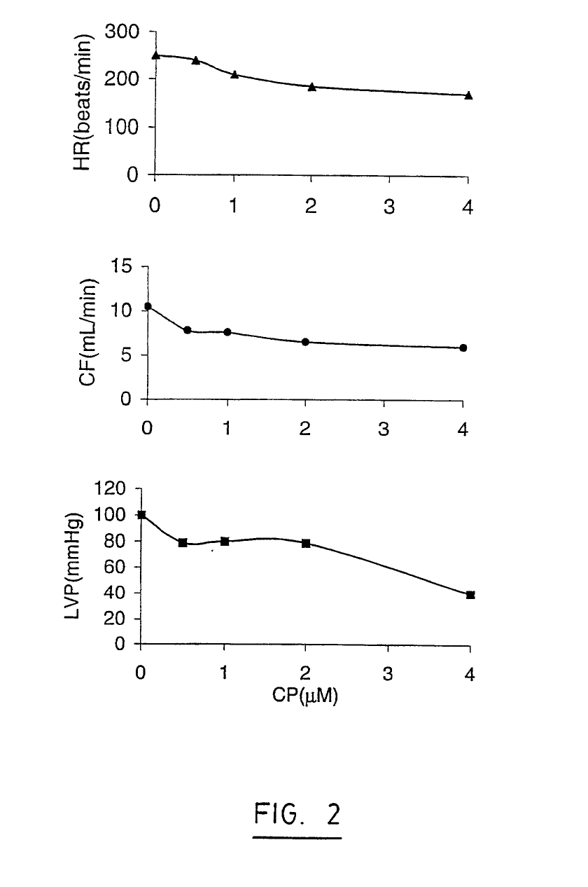

Ceruloplasmin, in concentrations of 1 to 2 μM was shown to protect isolated rat hearts against ischemia-reperfusion induced damage, while 4 μM was found to be cardiotoxic in this blood-free isolated heart model (Atanasiu et al, 1995). It was previously shown that CP is cardioprotective in concentrations up to 2 μM, while at concentrations of 4 μM and higher it presents an own cardiotoxic effect (Chahine et al., 1991; Atanasiu et al, 1995). The results of FIG. 2 obtained with isolated rat heart, indicates that the own cardiotoxicity of CP at concentrations at 2 μM-4 μM is still observed, even in the presence of TRIAD. However, these concentration values are physiologically encountered in vivo: indeed CP concentration in serum varies up to 300 μg/ml (2.4 μM) in normal conditions and can reach 700 μg/ml (5.3 μM) in acute inflammatory phases (Fox et al., 1995). It is supposed that differently to the blood-free system used herein, the blood flow will reduce the toxicity of CP at concentrations higher than 4 μM.

### 5. Conclusive Remarks

The association of TRIAD and CP appears to exert a strong antifibrillatory effect during reperfusion in the ischemic isolated rat heart, justifying further consideration of this association as a powerful protective agent against irreversible ventricular fibrillation, the most severe type of reperfusion-induced arrhythmias.

As low as 0.5 μM CP with small amounts of TRIAD (S2) (0.16×), completely protected hearts against the occurrence of irreversible fibrillations resulting from ischemia-reperfusion injury. This suggests that low concentrations of CP and TRIAD can efficiently assist heart in its efforts to assure its own protection. It is thus believed that higher efficiencies of protection would better be achieved with small concentrations of different antioxidants in association, than with saturating concentrations of a given one.

Finally, although the term “TRIAD” used herein refers to a composition comprising sodium pyruvate, vitamin E and egg yolk fatty acids, a person skilled in the art will understand that the compositions of the present invention are not restricted to these sole specific components as explained previously in the first part of the section “DETAILED DESCRIPTION OF THE INVENTION”.

### 6. REFERENCES

Throughout this paper, reference is made to a number of articles of scientific literature which are listed below:

Atanasiu, R., M. J. Dumoulin, R. Chahine, M. A. Mateescu, R. Nadeau (1995) Can. J. Physiol. Pharmacol., 73, 1253-1261.

Atanasiu, R., L. Gouin, M. A. Mateescu, R. Cardinal, R. Nadeau (1996) Can. J. Physiol. Pharmacol., 74, 652-656.

Atanasiu, R. L., Stéa, D., Mateescu, M. A., Vergely, C., Dalloz, F., Maupoil, V., Nadeau, R. and Rochette, L. (1998) Molec. Cell. Biochem., 189, 127-135.

Calabrese, L., M. A. Mateescu, M. Carbonaro, B. Mondovi (1988) Biochemistry International, 16, 199-208.

Chahine, R., Mateescu, M. A., Roger, S., Yamaguchi, N., De Champlain, J. and Nadeau, R. (1991) Can. J. Physiol. Pharmacol. 69, 1459-1464.

Chudej, L. L., Koke, J. R., Bittar, N. (1990) Cytobios 63, 41-53.

Dumoulin, M. J., R. Chahine, R. Atanasiu, R. Nadeau, M. A. Mateescu (1996) Arzneim. Forsch./Drug Res., 46, 855-861.

Fox, P. L., Mukhopadhyay, C., Ehrenwald, E. (1995) Life Sci. 56, 1749-1758.

Gokhale, M. S., Lin, J. R. and Yager, J. D. (1997) Toxicol. in Vitro 11, 753-759.

Gutteridge, G M and Stocks, J. (1981) Ceruloplasmin: physiological and pathological perspectives. Crit. Rev. Clin. Lab. Sci., 14. 257-329.

Martin, A. (1996) Dermatol. Surg. 22, 156-160.

Mateescu, M. A., R. Chahine, S. Roger, R. Atanasiu, N. Yamaguchi, G. Lalumière, R. Nadeau (1995) Arzneim. Forsch./Drug Res., 1995, 45, 476-80.

Mateescu, M. A., Chahine, R., Roger, S., Atanasiu, R., Yamaguchi, N., Lalumière,

G., Nadeau R., (1995) Arzneim. Forsch./Drug Res., 1995, 45, 476-80.

Mateescu, M. A., Wang, X. T., Befani, O., Dumoulin, M. J., Mondovi B., “Simultaneous chromatographic purification of ceruloplasmin and serum amineoxidase” in: Analytical and separation methods of Biomacromolecules (H. Aboul-Enein, Ed), Marcel Dekker Inc., New York 1999, p. 431-444.

Sheridan, J., Kern, E., Martin, A. and Booth, A. (1997) Antiviral Res. 36, 157-166.

Stevens, M. O., DiSilvestro, R. A., Harris, E. D. (1984) Biochemistry, 23, 261-266.

Wang, X. T., M. J. Dumoulin, O. Befani, B. Mondovi, M. A. Mateescu (1994) Preparative Biochemistry, 24, 237-50.

Of course, numerous modifications and improvements could be made to the embodiments that have been disclosed herein above. These modifications and improvements should, therefore, be considered a part of the invention.

What is claimed is:

1. A cardioprotective composition characterized in that it comprises a therapeutically effective amount of a mixture of pyruvate, at least one antioxidant, at least one lipid and ceruloplasmin and/or a functional derivative thereof.

2. The cardioprotective composition of claim 1, wherein said pyruvate, antioxidant, lipid and ceruloplasmin are present in an amount that have a synergistic cardioprotective effect on cardiac cells.

3. The cardioprotective composition of claim 1, wherein said at least one lipid consists of at least one fatty acid selected from the group consisting of monoglycerides, diglycerides, triglycerides, free fatty acids, and mixtures thereof.

4. The cardioprotective composition of claim 3, wherein said at least one fatty acid consist of a mixture of saturated and unsaturated fatty acids.

5. The cardioprotective composition of claim 4, wherein said fatty acids are present in an amount varying from about 0.001% v/v to about 1% v/v, by weight of the cardioprotective composition.

6. The cardioprotective composition of claim 1, wherein ceruloplasmin or its functional derivative is purified from blood using an one-step affinity chromatography on aminoethyl-agarose.

7. The cardioprotective composition of claim 1, wherein ceruloplasmin or its functional derivative is present in an in an amount varying from about 0.05 μM to about 10 μM.

8. The cardioprotective composition of claim 1, wherein pyruvate is selected from the group consisting of pyruvic acid, pharmaceutically acceptable salts of pyruvic acid, prodrugs of pyruvic acid, and mixtures thereof.

9. The cardioprotective composition of claim 1, wherein pyruvate is present in said composition in an amount varying from about 0.01 mM to about 100 mM.

10. The cardioprotective composition of claim 1, wherein said at least one antioxidant is selected from lipid-soluble antioxidants.

11. The cardioprotective composition of claim 1, wherein said at least one antioxidant is selected from the group consisting of Vitamin A, carotene, Vitamin E, pharmaceutically acceptable salts thereof, and mixtures thereof.

12. The cardioprotective composition of claim 1, wherein said at least one antioxidant is selected from the group consisting of Vitamin E, Vitamin E acetate and analogues of Vitamin E.

13. The cardioprotective composition of claim 1, wherein said at least one antioxidant is present in an amount varying from about 0.01 unit/ml to about 10 unit/ml of said composition.

14. The cardioprotective composition of claim 1, wherein it further comprises an agent selected from the group consisting of metal chelators, metal scavengers, proteinic metal chelators, proteinic scavengers, preserving agents, solubilizing agents, stabilizing agents, wetting agents, emulsifiers, salts, buffers and coating agents.

15. A method for treating a heart oxidative stress related condition comprising the administration to a patient in need thereof of a therapeutically effective amount of an antioxidative composition comprising pyruvate, at least one antioxidant, at least one lipid and ceruloplasmin and/or a functional derivative thereof.

16. A method for treating a heart oxidative stress related condition comprising:
administrating to a patient in need thereof of a therapeutically effective amount of an antioxidative composition comprising: pyruvate, at least one antioxidant and ceruloplasmin and/or a functional derivative thereof; and
providing into blood circulation of said patient at least one lipid having a synergistic therapeutic effect on cardiac cells in combination with said antioxidative composition.

17. The method of claim 16, wherein said at least one lipid is provided to said patient by increasing its lipidic blood level ratio through its diet.

18. The method of claim 16, wherein said heart oxidative stress related condition is an heart attack/failure, ischemic cardiopathy, or handling an heart before and during an heart transplantation.

19. A method for the treatment of cardiac cells, comprising contacting said cells with a therapeutically effective amount of an antioxidative composition comprising pyruvate, at least one antioxidant, at least one lipid and ceruloplasmin and/or a functional derivative thereof.

20. The method of claim 19, for protecting cardiac cells in vitro, in vivo and ex vivo against an oxidative stress related condition.

21. The method of claim 19, for the treatment of heart attack/failure, the treatment of ischemic cardiopathy, the conservation of heart before and during transplantation, or the treatment heart oxidative stress related condition(s).

22. A method for preparing a cardioprotective composition, comprising the steps of:
a) providing a therapeutically effective amount of i) pyruvate, ii) at least one antioxidant, iii) at least one lipid, and iv) ceruloplasmin and/or a functional derivative thereof; and
b) mixing together the components i), ii), iii) and iv) of step a) in a physiological buffered saline solution to obtain a homologous pharmaceutically acceptable suspension.
